(19) United States
(12) Patent Application Publication
(10) Pub. No.: US 2024/0315727 A1
(43) Pub. Date: Sep. 26, 2024

(54) IDENTIFICATION OF ELASTIC LAMINA TO GUIDE INTERVENTIONAL THERAPY

(71) Applicant: Avinger, Inc., Redwood City, CA (US)

(72) Inventors: John B. SIMPSON, Woodside, CA (US); Xuanmin HE, Sunnyvale, CA (US); Ryan RADJABI, Campbell, CA (US); Richard R. NEWHAUSER, Redwood City, CA (US)

(21) Appl. No.: 18/582,526

(22) Filed: Feb. 20, 2024

Related U.S. Application Data
(63) Continuation of application No. 17/075,548, filed on Oct. 20, 2020, now Pat. No. 11,944,342.
Application No. 17/075,548 is a continuation of application No. 16/194,183, filed on Nov. 16, 2018, now Pat. No. 10,806,484.
Application No. 16/194,183 is a continuation of application No. 14/899,893, filed on Dec. 18, 2015, now Pat. No. 10,130,386.
(60) Provisional application No. 61/843,866, filed on Jul. 8, 2013.

(51) Int. Cl. A61B 17/3207; A61B 17/00; A61B 17/22; A61B 34/10; A61B 90/00

(52) U.S. Cl. CPC A61B 17/3207; A61B 17/320783; A61B 90/361; A61B 2017/00119; A61B 2017/22071; A61B 2017/320791; A61B 2034/107; A61B 2090/08021; A61B 2090/3735

(57) ABSTRACT

Described herein are systems and methods for identifying elastic lamina during interventional procedures, such as atherectomy. Such identification can be used to avoid trauma to the external elastic lamina during the procedure. Systems and methods may use a catheter having on-board imaging and may be configured to gather optical coherence tomography (OCT) images.

### CROSS REFERENCE TO RELATED APPLICATIONS

This application is a continuation of U.S. patent application Ser. No. 17/075,548, filed on Oct. 20, 2020, titled “IDENTIFICATION OF ELASTIC LAMINA TO GUIDE INTERVENTIONAL THERAPY,” now U.S. Publication No. US-2021-0267621-A1, which is a continuation of U.S. patent application Ser. No. 16/194,183, filed on Nov. 16, 2018, titled “IDENTIFICATION OF ELASTIC LAMINA TO GUIDE INTERVENTIONAL THERAPY,” now U.S. Pat. No. 10,806,484, which is a continuation of U.S. patent application Ser. No. 14/899,893, filed on Dec. 18, 2015, titled “IDENTIFICATION OF ELASTIC LAMINA TO GUIDE INTERVENTIONAL THERAPY,” now U.S. Pat. No. 10,130,386, which is a 371 of International Patent Application No. PCT/US2014/045799, filed on Jul. 8, 2014, titled “IDENTIFICATION OF ELASTIC LAMINA TO GUIDE INTERVENTIONAL THERAPY,” now International Publication No. WO 2015/006353, which claims priority to U.S. Provisional Patent Application No. 61/843,866, titled “IDENTIFICATION OF ELASTIC LAMINA TO GUIDE INTERVENTIONAL THERAPY,” filed on Jul. 8, 2013, each of which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Referring to FIG. 1, a normal healthy artery wall includes layers of tissue, such as the innermost intima 111, the media 107, the adventitia 103, and the periadventitia 101. The intima 111, media 107, and adventitia 103 are separated by two elastic membranes. The inner membrane is the internal clastic lamina (IEL) 109, which separates the intima 111 from the media 107, and the outer membrane is the external elastic lamina (EEL) 105, which separates the media 107 from the adventitia 103.

Coronary artery disease (CAD) and Peripheral artery disease (PAD) are both caused by the progressive narrowing of the blood vessels most often caused by atherosclerosis, the collection of plaque or a fatty substance along the inner lining or intima of the artery wall. Over time, this substance hardens and thickens, which can cause an occlusion in the artery, completely or partially restricting flow through the artery. Blood circulation to the arms, legs, stomach and kidneys brain and heart may be reduced, increasing the risk for stroke and heart disease.

Peripheral artery disease (PAD) and coronary artery disease (CAD) affect millions of people in the United States alone. PAD and CAD are silent, dangerous diseases that can have catastrophic consequences when left untreated. CAD is the leading cause of death in the United States while PAD is the leading cause of amputation in patients over 50 and is responsible for approximately 160,000 amputations in the United States each year.

Interventional treatments for CAD and PAD may include endarterectomy and/or atherectomy. Endarterectomy is surgical removal of plaque from the blocked artery to restore or improve blood flow. Endovascular therapies such as atherectomy are typically minimally invasive techniques that open or widen arteries that have become narrowed or blocked. Other treatments may include angioplasty to open the artery. For example, a balloon angioplasty typically involves insertion of a catheter into a leg or arm artery and positioning the catheter such that the balloon resides within the blockage. The balloon, connected to the catheter, is expanded to open the artery. Surgeons may then place a wire mesh tube, called a stent, at the area of blockage to keep the artery open.

During interventional treatments, trauma often occurs to the IEL 109, media 107, EEL 105, and adventitia 103. Trauma to the EEL 105 and/or adventitia 103 can initiate a severe inflammatory response, which can accelerate scarring and cause potential closure of the vessel. Disruption of the EEL 105 can also signal complimentary and inflammatory factors that accelerate and further promote restenosis. Accordingly, an interventional treatment that avoids trauma to EEL 105, and thus to the adventitia 103, is desired.

### SUMMARY OF THE DISCLOSURE

Described herein is a system and method for identifying elastic lamina during interventional procedures and treatments. Such identification can be used to avoid trauma to the external elastic lamina during such procedures and treatments.

In general, in one embodiment, a method of performing atherectomy includes: (1) inserting an atherectomy device into a vessel; (2) gathering optical coherence tomography (OCT) images using an imaging sensor on the device; (3) identifying an external clastic lamina in the OCT images; and (4) cutting tissue in the vessel based upon the identification.

This and other embodiments can include one or more of the following features. The OCT images can be a toroidal view of the vessel. Identifying an external elastic lamina can include identifying an outer-most bright line in the toroidal view. Cutting tissue in the vessel based upon the identification can include adjusting a depth of cut based upon the identification. Cutting tissue in the vessel based upon the identification can include reorienting a distal tip of the device based upon the identification. Cutting tissue in the vessel can include cutting right up to the external clastic lamina, but not through the external clastic lamina. The identification can be performed automatically. Adjusting the depth of cut can include moving the cutter from an active mode to a passive mode. Moving the cutter from an active mode to a passive mode can include at least partially deflating a balloon on the device. The adjusting step can be performed automatically. The reorienting step can include using a marker in the OCT images to reorient the tip. The method can further include determining a distance between the cutter and the external clastic lamina. The method can further include activating an alarm if the distance is below a threshold value. The method can further include stopping the cutting if the distance is below a threshold value. The method can further including highlighting the external lamina in the OCT images after the identifying step.

In general, in one embodiment, an atherectomy system includes a catheter having an OCT imaging sensor attached thereto configured to gather OCT images and a controller. The controller is configured to automatically identify an external elastic lamina in the OCT images.

This and other embodiments can include one or more of the following features. The system can further include a display connected to the controller, and the display can be configured to display the OCT images as a toroidal view of the vessel. The controller can be further configured to highlight the external elastic lamina in the OCT images on the display after identification. The controller can be further configured to adjust a depth of cut based upon the identification. Adjusting a depth of cut can include moving the cutter from an active mode to a passive mode. Moving the cutter from an active mode to a passive mode can include at least partially deflating a balloon on the device. The controller can be further configured to reorient a distal tip of the device based upon the identification. The controller can be further configured to determine a distance between the cutter and the external elastic lamina. The controller can be configured to activate an alarm if the distance is below a threshold value. The controller can be configured to prevent cutting if the distance is below a threshold value.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a histological view of a healthy vessel.

FIG. 2 is a view of an exemplary catheter with on-board imaging.

FIGS. 3a-3ii are exemplary OCT images wherein the internal elastic lamina and/or the external elastic lamina can be identified.

FIGS. 4a and 4b are OCT images taken with an imaging atherectomy device having an inflatable balloon to urge the cutter against the wall. FIG. 4a shows an OCT image during cutting into the media, close to the EEL and adventitia with the balloon fully inflated. FIG. 4B shown an OCT image where the balloon has been deflated to reduce the cutting depth and avoid the EEL and adventitia.

FIGS. 5a and 5b are OCT images taken with a directional atherectomy device. FIG. 5a shows the direction of cut directly towards the artery wall structure with media and EEL. FIG. 5b shows adjustment of the direction away from the artery wall and towards plaque.

FIGS. 6a-6h show exemplary tissue excised with the identification methods described herein and the OCT images taken during cutting procedures.

FIGS. 7a-7e show a method of automatic detection of the EEL with a controller.

FIG. 8 shows an OCT image with a highlighted band on the EEL.

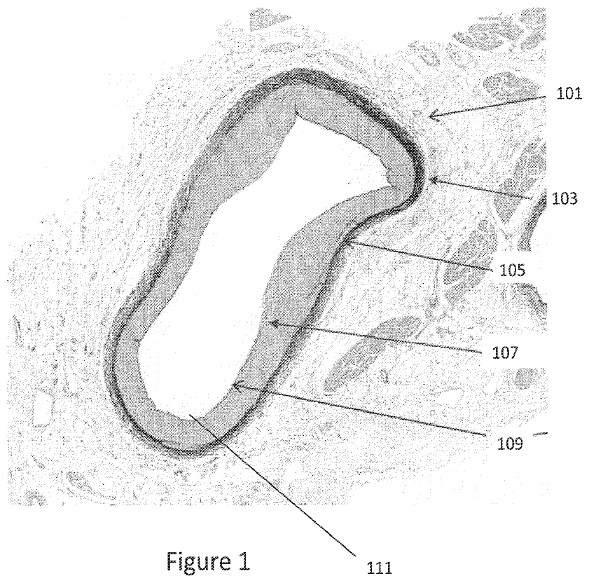

### DETAILED DESCRIPTION

Described herein is a system and method for identifying elastic lamina during interventional treatments using a catheter having on-board imaging.

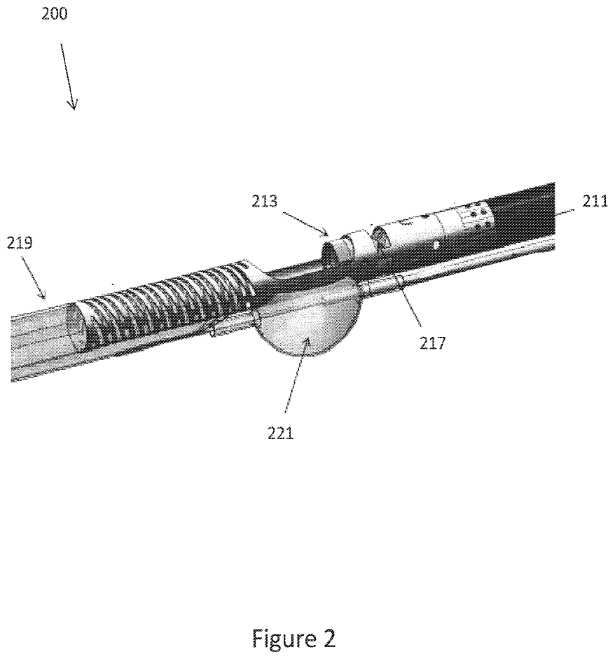

Referring to FIG. 2, an interventional catheter 200, such as an atherectomy catheter or an occlusion-crossing catheter, can include an elongate body 211 with a cutter 213 extending therefrom. An imaging sensor 217 can be configured to gather optical coherence tomography (OCT) images. A distal nosecone 219 can be configured to collect tissue cut by the cutter 213. In some embodiments, the device 200 can be a directional atherectomy device, and a balloon 221 can be used to expose the cutter 213 and/or urge the cutter 213 against the vessel wall for cutting. The amount of inflation of the balloon 212 can be varied to modify a depth of cut made by the cutter 213 into the vessel wall. Exemplary atherectomy devices are described further in U.S. patent application Ser. No. 12/829,277, filed Jul. 1, 2010, titled “ATHERECTOMY CATHETER WITH LATERALLY-DISPLACEABLE TIP,” now U.S. Patent Application Publication No. 2011/0004107, U.S. patent application Ser. No. 13/175,232, filed Jul. 1, 2011, titled “ATHERECTOMY CATHETERS WITH LONGITUDINALLY DISPLACEABLE DRIVE SHAFTS,” now U.S. Patent Application Publication No. 2012/0046679, U.S. patent application Ser. No. 13/654,357, filed Oct. 17, 2012, titled “ATHERECTOMY CATHETERS AND NON-CONTACT ACTUATION MECHANISM FOR CATHETERS,” now U.S. Patent Application Publication No. 2013/0096589, International Patent Application No. PCT/US2013/031901, filed Mar. 15, 2013, titled “ATHERECTOMY CATHETERS WITH IMAGING,” now published as WO 2013/172970, and International Patent Application No. PCT/US2013/032494, filed Mar. 15, 2013, titled “BALLOON ATHERECTOMY CATHETERS WITH IMAGING,” now published as WO 2014/039099, the entireties of which are incorporated by reference herein.

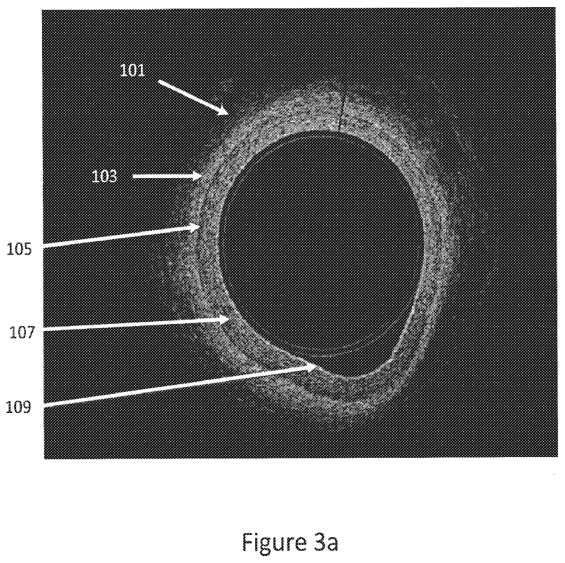

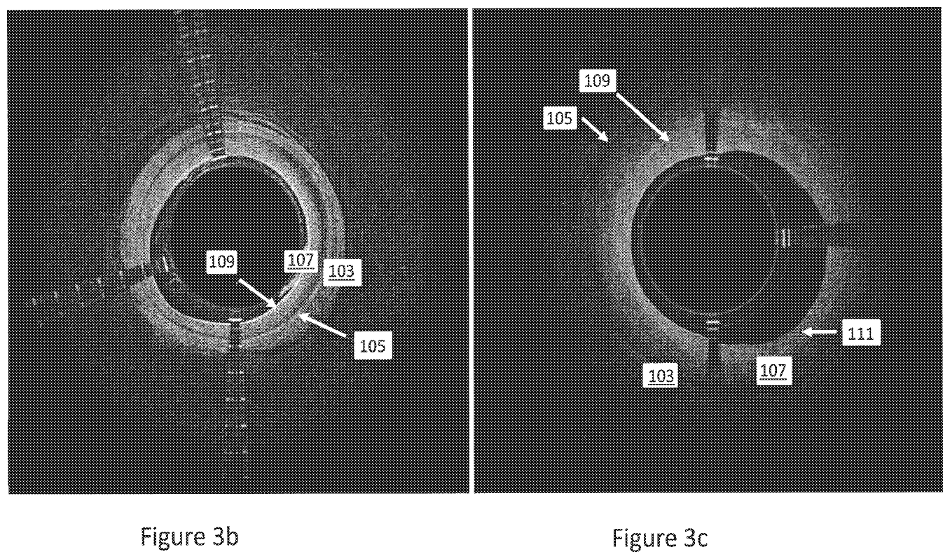

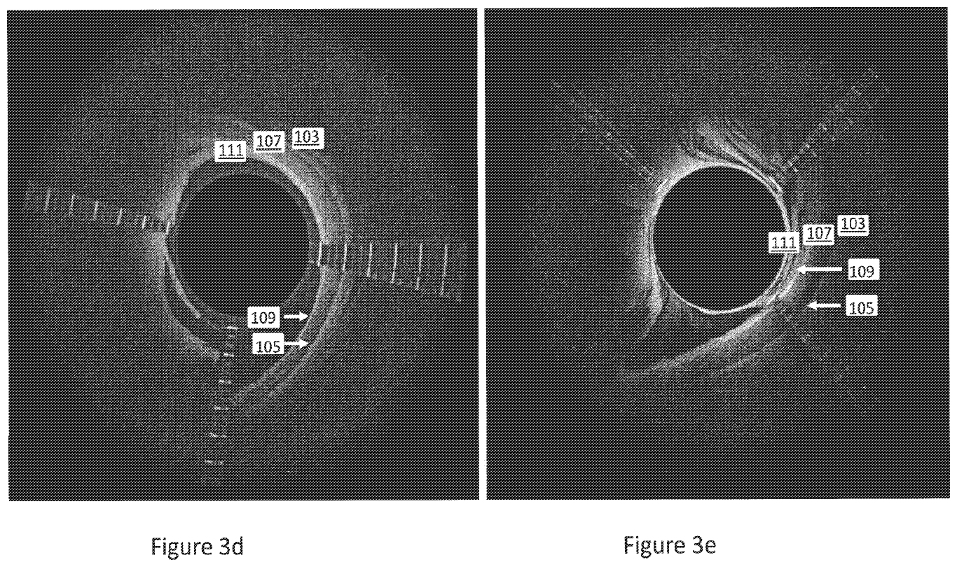

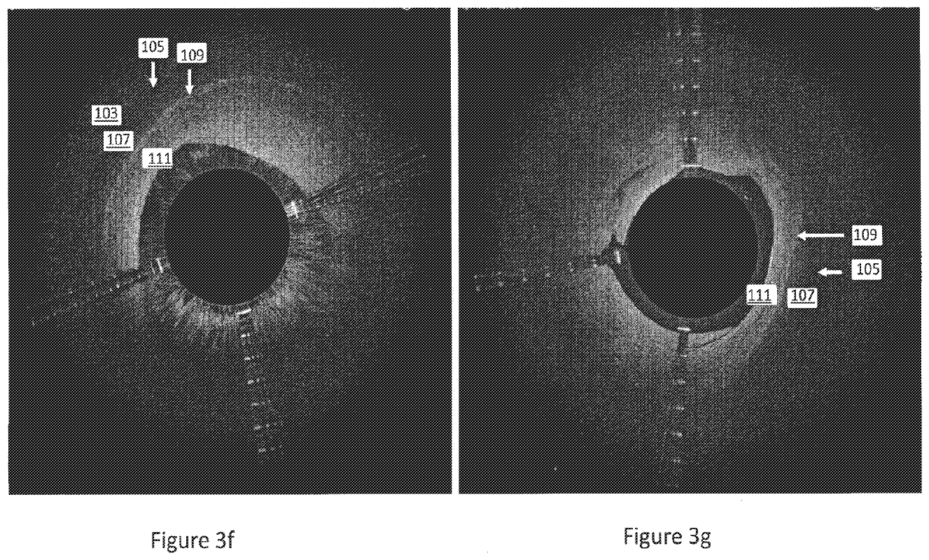

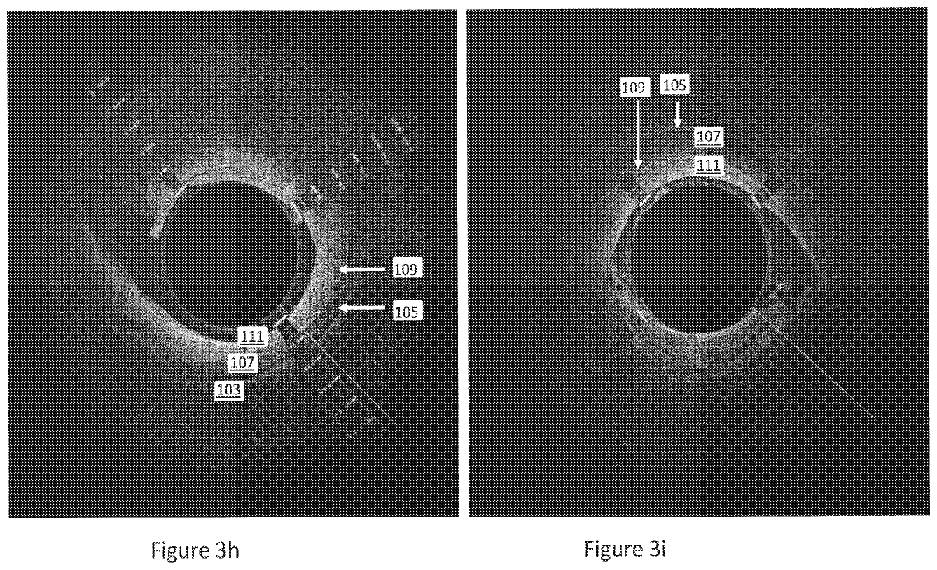

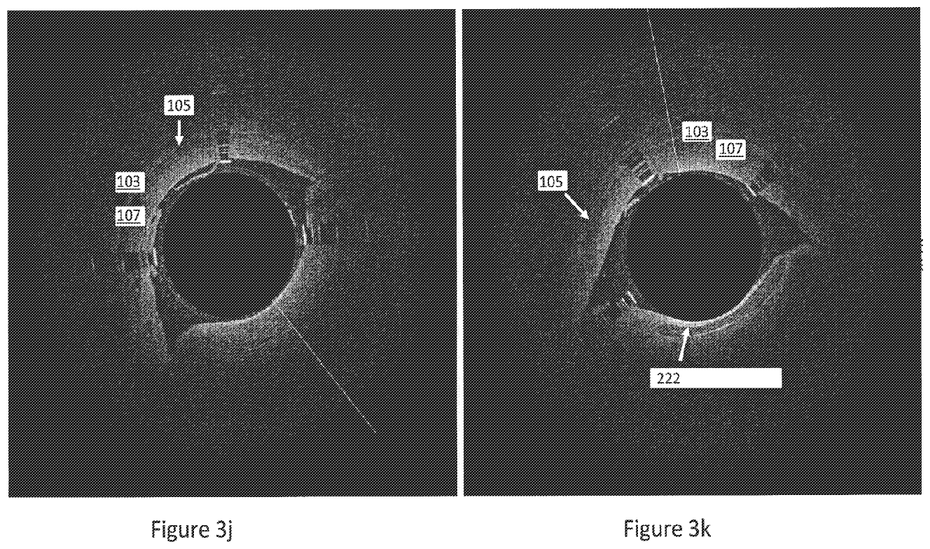

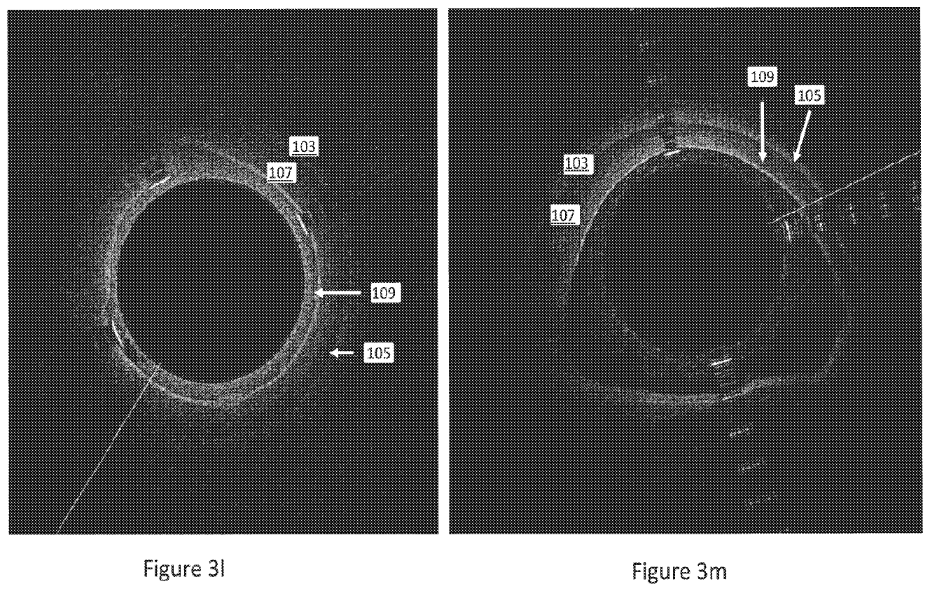

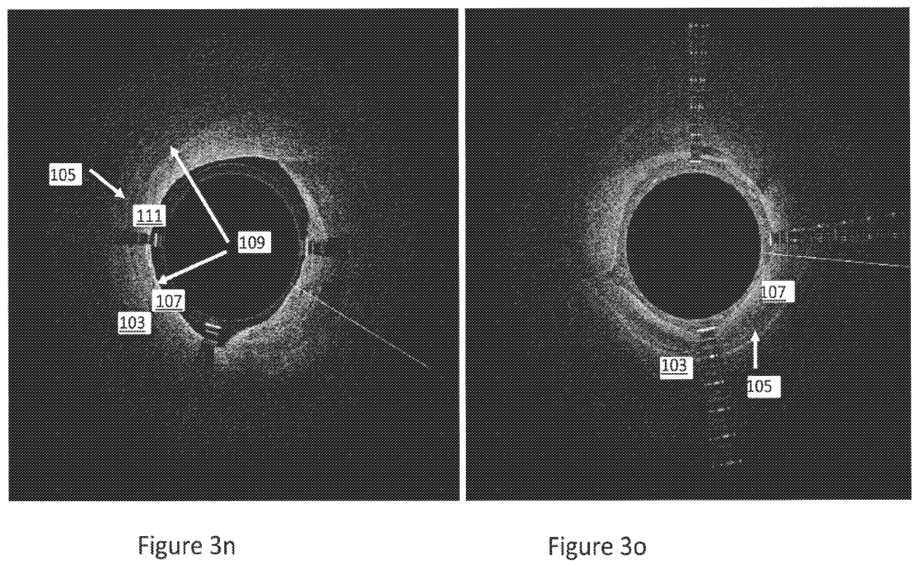

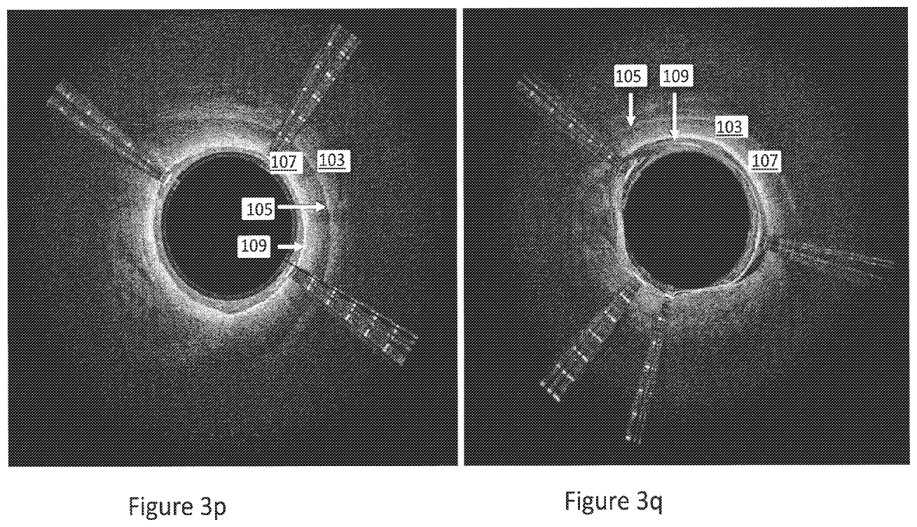

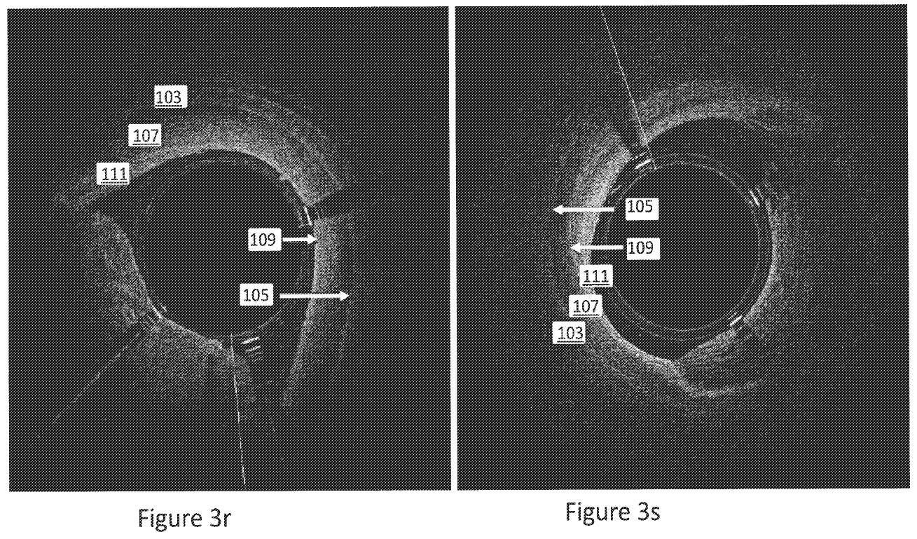

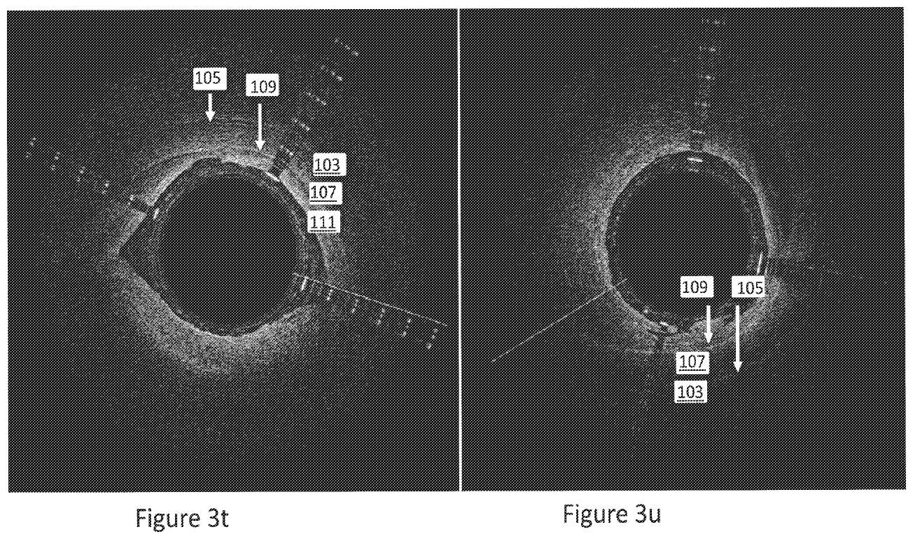

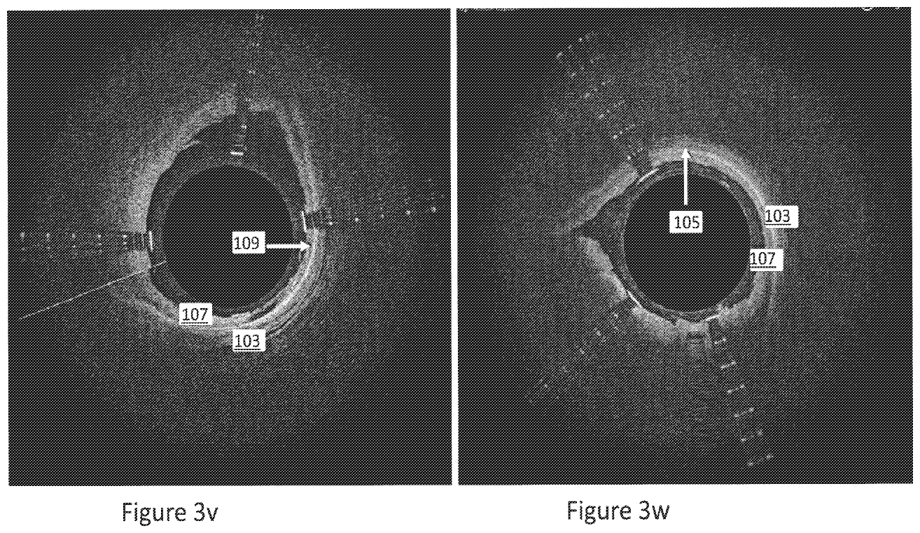

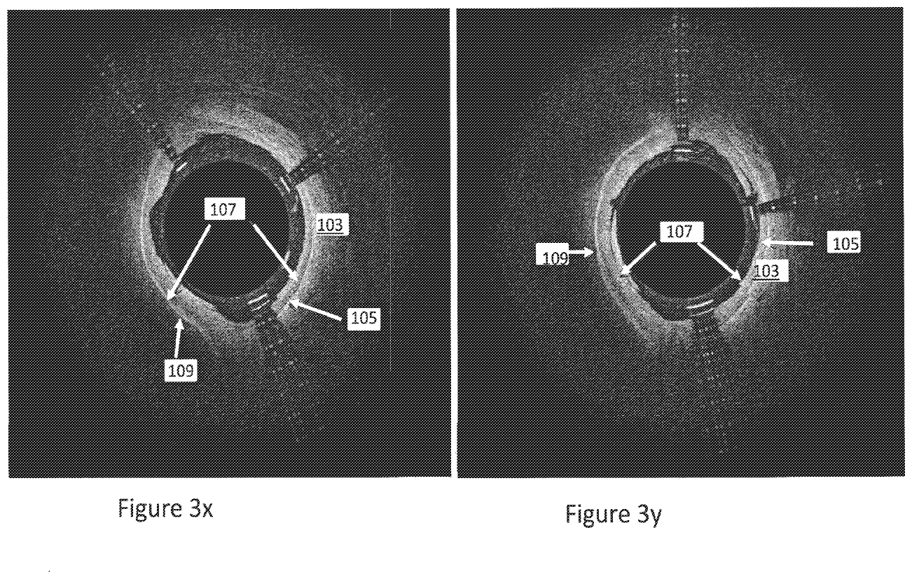

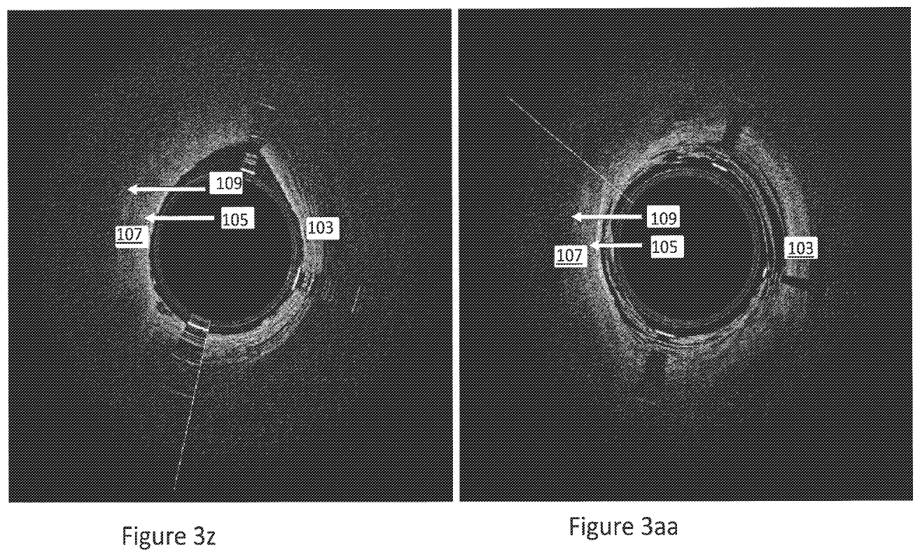

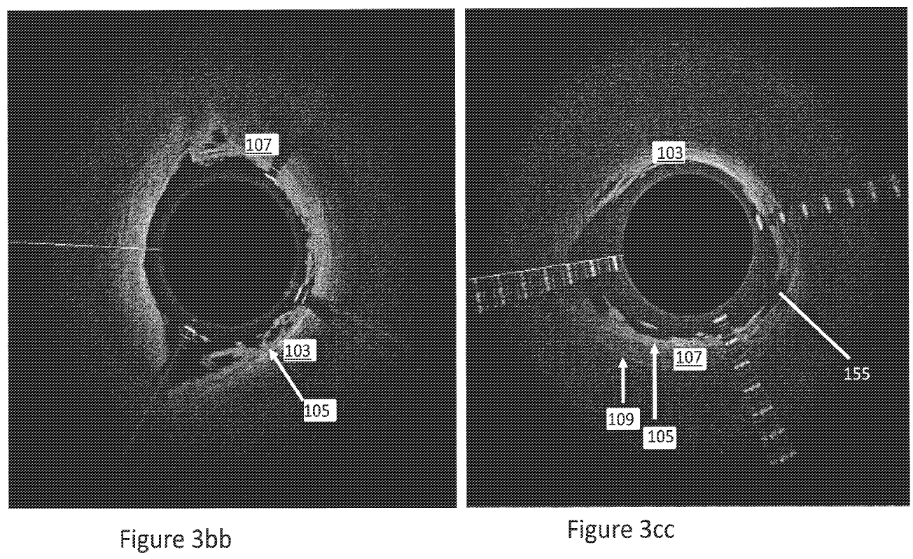

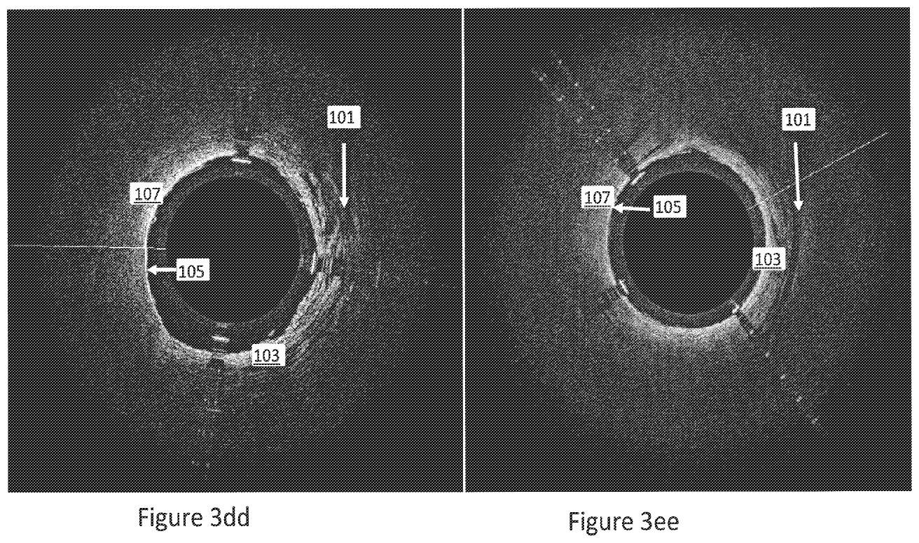

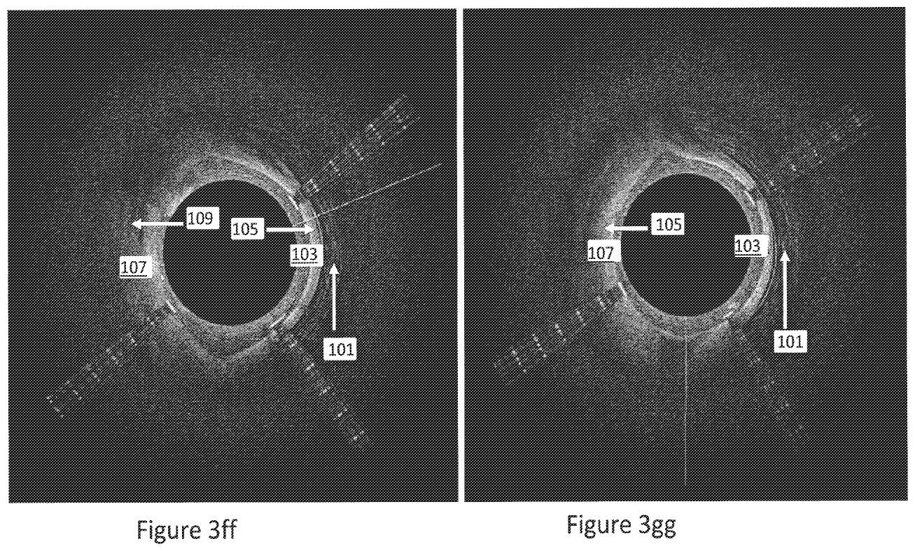

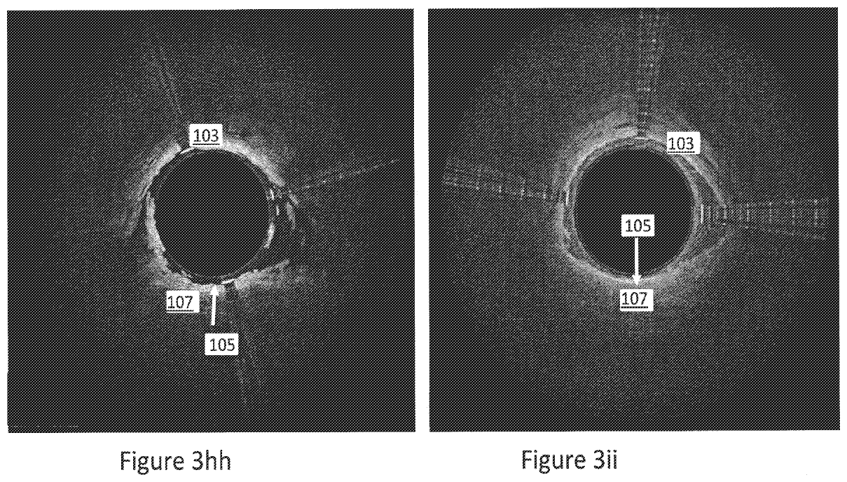

Referring to FIGS. 3a-3ii, OCT images obtained with such an on-board imaging catheter can show the walls of the vessel, such as in a toroidal view of the walls. The resulting images can, for example, show plaque or thrombosis as well as layers of the vessel, including the backscattering or signal-rich intima 111, the media 107 that frequently has low backscattering or is signal-poor, the heterogeneous and frequently high backscattering adventitia 103, and/or the periadventitial tissue 101 characterized by large clear structures. The OCT image can further advantageously clearly show the IEL 109 as a distinct line between the media 107 and the intima 111 and the EEL 105 as a distinct line between the media 107 and the adventitia 103. For example, the IEL 109 and EEL 105 can be displayed as thin bright structures (i.e., be highly backscattering). In many cases, the IEL 109 and EEL 105 appear as continuous lines following along (substantially parallel with) the internal and external perimeter of the media 107.

The OCT images collected with the catheter can thus be used to clearly identify the EEL 105. Moreover, OCT advantageously has a higher resolution than other types of imaging, such as ultrasound, thereby allowing for the clear identification of the EEL 105. Further, upon identification the EEL 105 in the images, the interventional therapy (e.g., atherectomy) can be tailored so as to avoid or limit interaction with the EEL 105 and adventitia 103, thereby avoiding the inflammatory response that occurs if the EEL 105 or adventitia 103 are injured.

Referring to FIGS. 4a and 4b, in one embodiment, the depth of cut with the atherectomy catheter can be adjusted based upon the identification of the EEL 105, such as by deflating a balloon or otherwise adjusting the cutting depth, in order to avoid excising the EEL 105 or the adventitia 103. For example, FIG. 4a shows an OCT image where the balloon 221 is fully inflated. The resulting cut (the cutter position is indicated by the dark circle in the center and the direction is opposite to the middle marker 333) is close to the media 107 and EEL 105. In contrast, in FIG. 4b, the balloon 221 has been deflated in order to pull the cutter away and leave a space 166 between the cutter and the EEL 105 (note that FIG. 4b shows the cutting mark 177, close to the EEL 105, created by the cut of FIG. 4a).

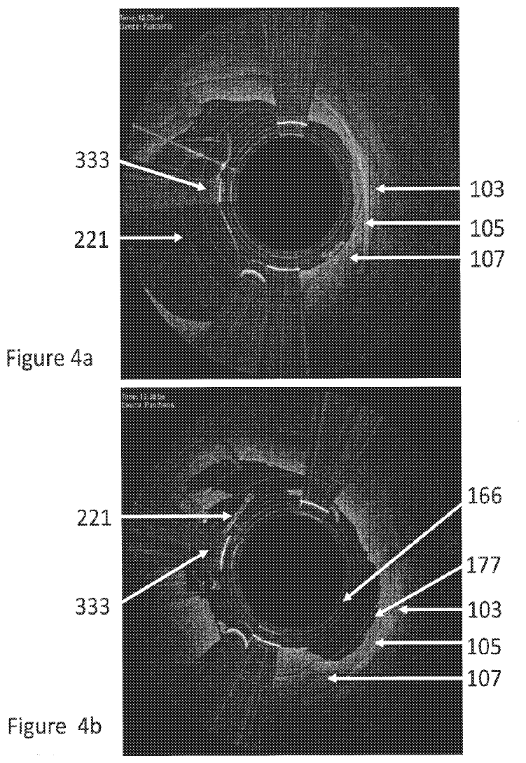

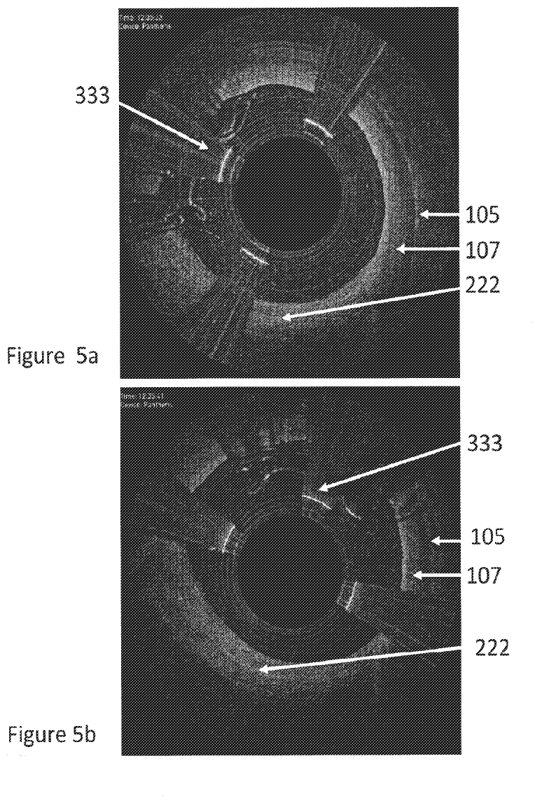

Referring to FIGS. 5a-5b, in one embodiment, the direction of the cut can be modified to avoid the EEL 105, i.e., the distal tip of the catheter 200 can be oriented away from the EEL 105. In some embodiments, markers on the image can be used to help orient the direction of the catheter to ensure that the EEL is not cut (i.e., the middle marker 333 can show a position directly opposite to the direction of the cut). The cutting direction can thus be changed from being oriented directly at the healthy artery wall (and thus normal to the EEL) and instead towards the plaque 333.

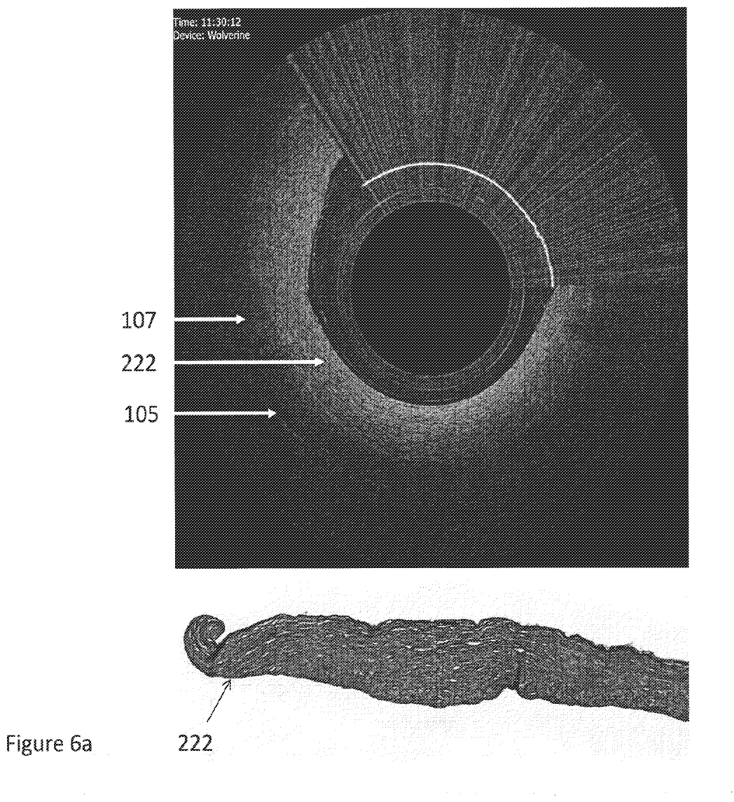

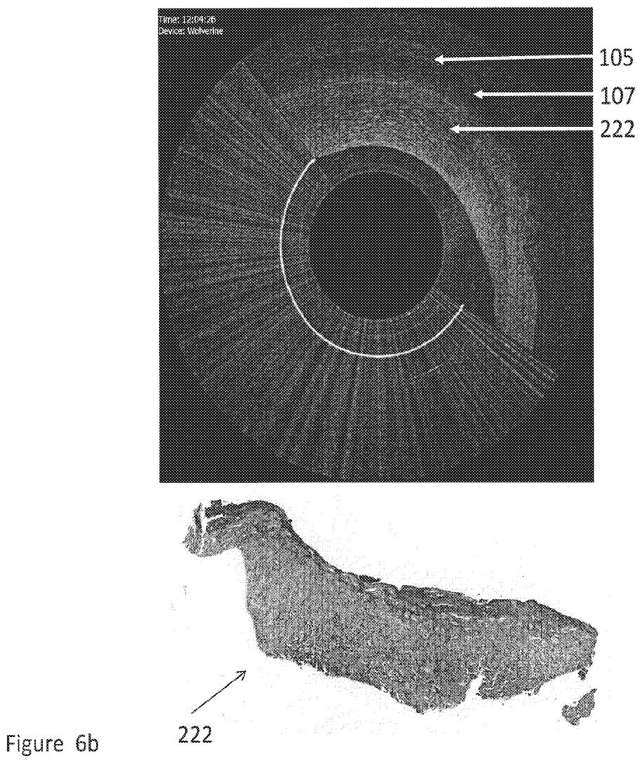

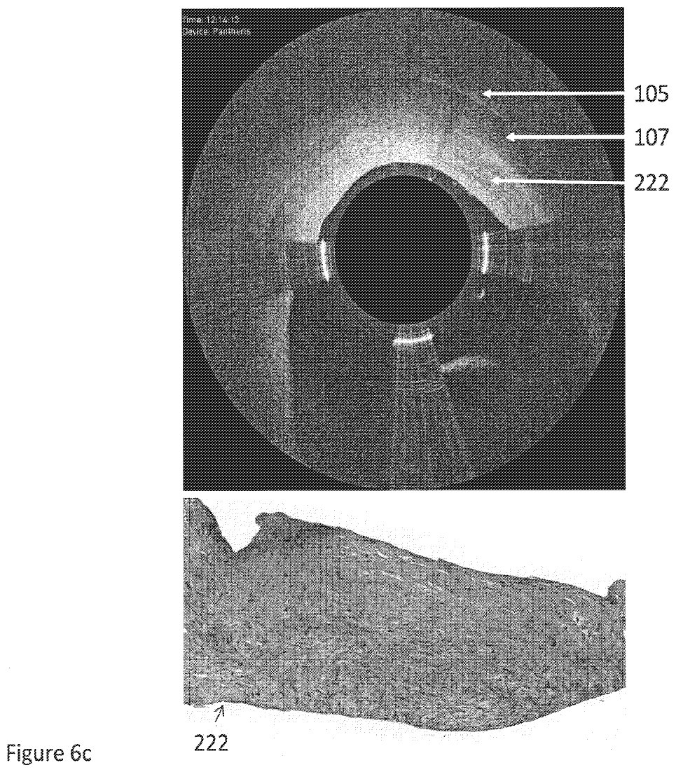

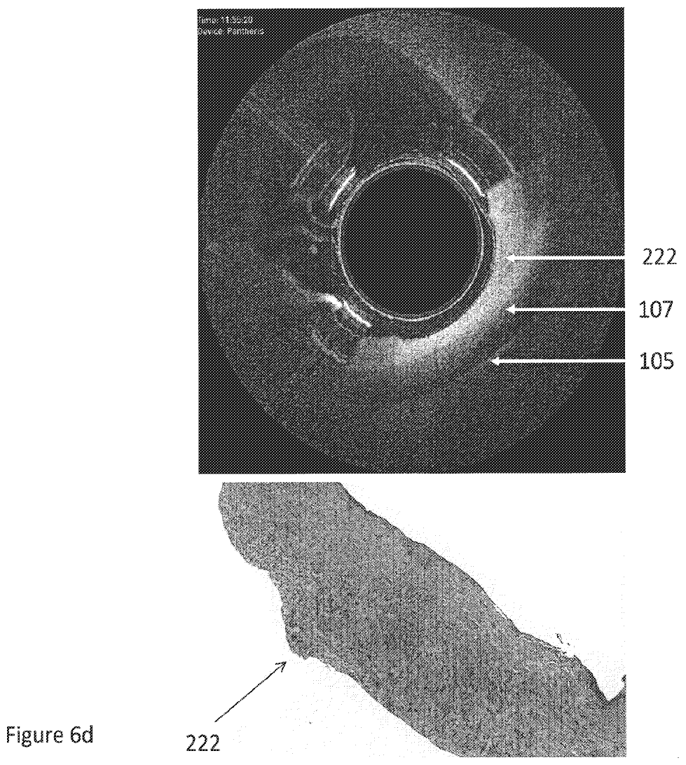

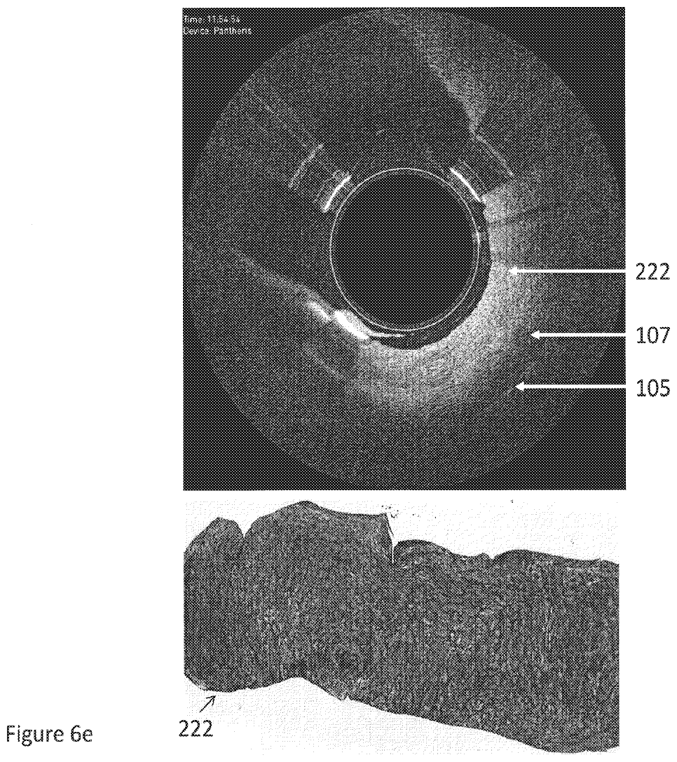

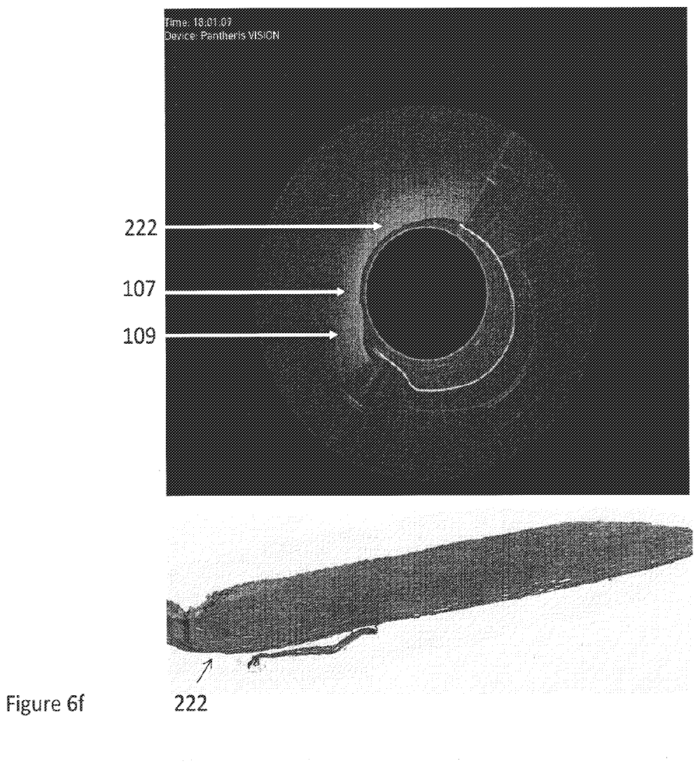

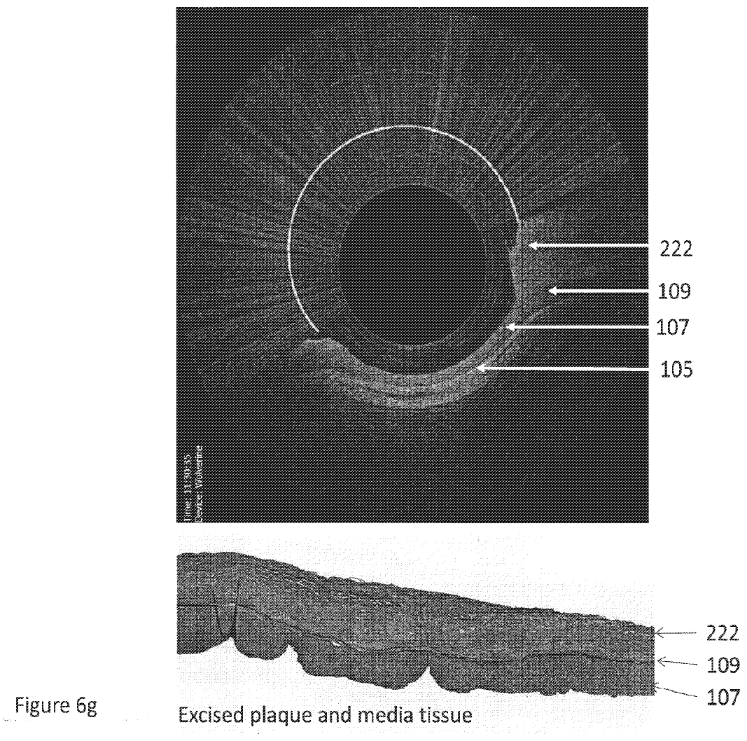

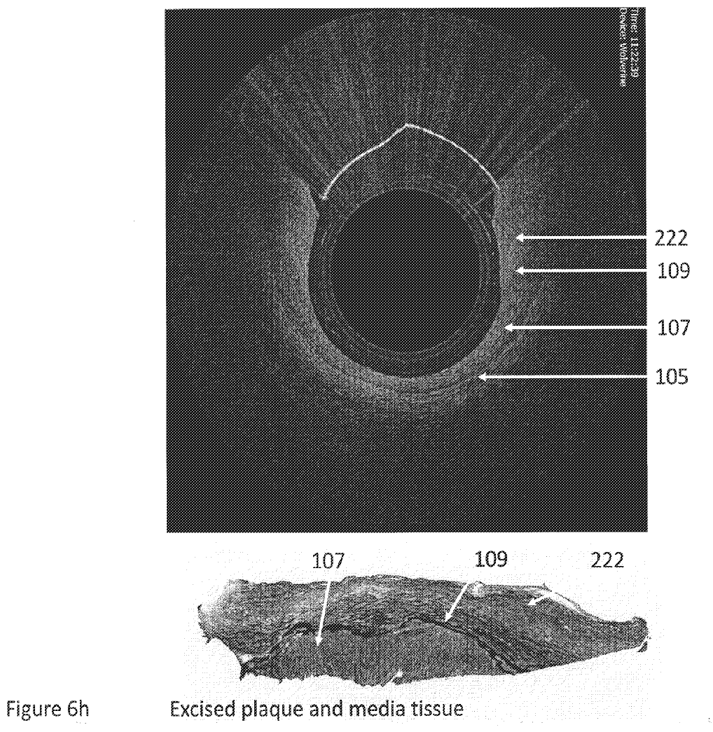

As a result of the direction and/or depth modifications, the tissue can be cut right up to, but not through, the EEL 105. FIGS. 6a-6h show the OCT image and resulting tissue cut using the identification methods described herein. As shown, only plaque tissue 222 (and the IEL 109 and media 107 in FIG. 6g) is cut, but not the EEL 105.

Moreover, if the OCT images show that trauma has occurred (i.e., if the images shown a break 155 in the continuity of the bright lines as shown in FIG. 3cc), the treatment plan can be adjusted so as to avoid further trauma, such as by reorienting the catheter or adjusting the depth of cut. Furthermore, if trauma to the EEL 105 is identified, the treatment plan can be adjusted to mitigate the inflammatory cascade by implanting drug coated stents, drug eluting balloons, or oral medication (e.g., anti-inflammatory, Plavix, etc.).

In some embodiments, the identification of the EEL 105 can be performed manually by a physician or technician viewing the imagines.

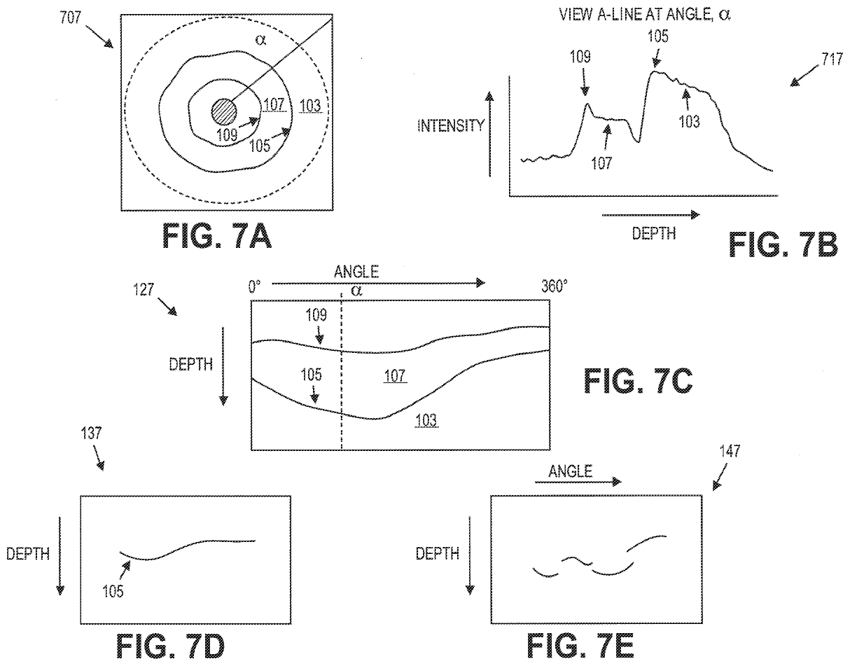

In other embodiments, the identification of the EEL 105 can be performed automatically with a controller. For example, referring to FIGS. 7a-7e, an A-line graph 717 of intensity vs. depth can be taken at an angle α within an OCT image 707. The peak intensity in the graph will correspond to the EEL 105 while the next highest peak will correspond to the IEL 109. The controller can then move to a new angle and search for the contour. For example, if the if the sector is unfolded into a B-scan 127, the EEL 105 can be identified at the set angle α and then the process repeated to find a continuous edge. As shown in FIG. 7d, if the line is continuous, it signifies the EEL 105. If it is discontinuous, then either the features is not the EEL or the EEL has been broken.

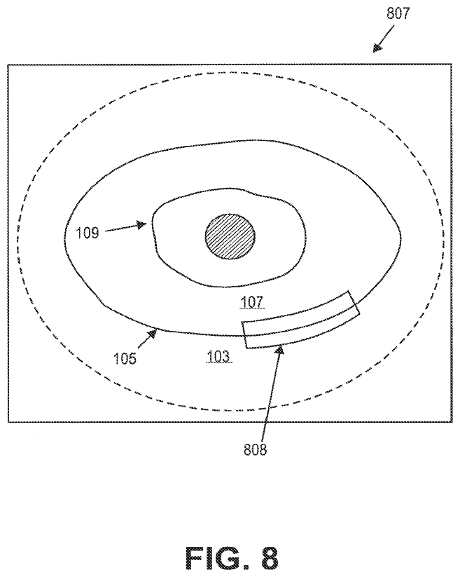

In some embodiments, the EEL can be automatically labeled or highlighted in the display of the OCT images. Referring to FIG. 8, the EEL 105 could be labeled in the OCT image 807, for example, with a transparent colored band 808. The band 808 can, for example, be brighter for greater confidence in EEL detection. The band 808 can further include different colors or widths, can be overlaid with different small shapes (such as dots or triangles). Further, the band 808 can be drawn outside of the OCT image 807 to identify where the feature is without interrupting the viewing (e.g., as an arc outside the OCT region).

Further, in some embodiments, a controller can use the identification of the EEL 105 to automatically assist with the interventional procedure. That is, in some embodiments, both the EEL 105 can be detected as well as the distance between the EEL and the cutter edge. The controller can thus calculate a distance between the EEL and the cutter and take a set action if that distance goes below a threshold value. For example, the controller can set off an alarm (e.g., audible noise, flash of light, graphic symbol). In other embodiments, the controller can shut down the cutter activation if the distance is below the threshold (and/or if the EEL 105 is going to be or has been broken or damaged as shown in FIG. 7c). In still other embodiments, the amount of urge on the catheter can be automatically reduced (such as the amount of balloon inflation). To automatically reduce the amount of urge, the inflation/deflation of the balloon, and thus the movement of the cutter between an active (or open) position to a passive (or closed) position can be automated with servos (or motors, or actuators) that are controlled by the controller. The controller can thus use the identification of the EEL 105 to partially or completely deflate the balloon, thereby reducing the cut depth and/or moving the cutter from an active to a passive position.

By identifying the EEL in images taken during interventional therapy, injury or trauma to those structures can advantageously be avoided. For example, referring to FIGS. 6a-4h, tissue cut during an atherectomy procedure using such techniques can advantageously include substantially only the diseased thrombosis or plaque (little to no media 107, adventitia 103, or EEL 105). In contrast, in atherectomy procedures where such methods are not used, excised tissue can include EEL 105, media 107, and/or adventitia 103, in addition to the diseased tissue, suggesting that an enhanced inflammatory response was instigated during the atherectomy procedure.

Additional details pertinent to the present invention, including materials and manufacturing techniques, may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are a plurality of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

1. (canceled)

2. A catheter system, comprising:
a catheter having an OCT imaging sensor attached thereto configured to gather OCT images; and
a controller, the controller configured to automatically identify an external elastic lamina in the OCT images.

3. The system of claim 2, further comprising a display connected to the controller, the display configured to display the OCT images as a toroidal view of the vessel.

4. The system of claim 3, wherein the controller is further configured to highlight the external elastic lamina in the OCT images on the display.

5. The system of claim 2, wherein the controller is further configured to adjust a depth of cut based upon the identification.

6. The system of claim 5, wherein the controller is configured to move a cutter of the catheter from an active mode to a passive mode to adjust the depth of cut.

7. The system of claim 6, wherein the catheter further includes a balloon configured to inflate or deflate to expose or cover the cutter, and wherein the controller is configured to at least partially deflate the balloon to move the cutter from the active mode to the passive mode.

8. The system of claim 2, wherein the controller is further configured to reorient a distal tip of the catheter based upon the identification.

9. The system of claim 2, wherein the controller is further configured to determine a distance between a cutter of the catheter and the external elastic lamina.

10. The system of claim 9, wherein the controller is configured to activate an alarm if the distance is below a threshold value.

11. The system of claim 9, wherein the controller is configured to prevent cutting if the distance is below a threshold value.
